(19) United States
(12) Patent Application Publication
(10) Pub. No.: US 2024/0216912 A1
(43) Pub. Date: Jul. 4, 2024

(54) MICROFLUIDIC DEVICE

(71) Applicant: Hewlett-Packard Development Company, L.P., Spring, TX (US)

(72) Inventors: Michael Cumbie, Corvallis, OR (US); Viktor Shkolnikov, Palo Alto, CA (US); Chien-Hua Chen, Corvallis, OR (US)

(73) Assignee: Hewlett-Packard Development Company, L.P., Spring, TX (US)

(21) Appl. No.: 18/557,567

(22) PCT Filed: Apr. 30, 2021
(86) PCT No.: PCT/US2021/030076

§ 371 Date: Oct. 26, 2023

(51) Int. Cl. B01L 3/00

(52) U.S. Cl. CPC B01L 3/502715; B01L 3/502707; B01L 2200/025; B01L 2200/12; B01L 2300/0663; B01L 2300/12; B01L 2300/1805

(57) ABSTRACT

A microfluidic device is described. The microfluidic device comprises a reaction chamber and a temperature control module overlaying the reaction chamber. The temperature control module comprises a temperature regulating flow channel disposed between two layers and in thermal contact with the reaction chamber, and wherein at least a portion of each layer comprises an optically transparent material. Also described is a method of manufacturing a microfluidic device and a method of controlling temperature of a reaction in a microfluidic device.

### BACKGROUND

Microfluidic devices generally include one or more microfluidic flow channels connecting other components such as fluid inlets and outlets, sample preparation zones, reaction chambers, and detection zones. Other components can include microprocessing circuitry for flow and thermal control.

The Polymerase Chain Reaction (PCR) is used to amplify specific nucleic acid sequences and detect their presence in a sample. PCR can be used for many different applications, including quantification of gene expression, patient genotyping and also as a diagnostic tool to identify the presence of one or more pathogens, for example bacteria or viruses in a sample from a patient by amplifying and detecting nucleic acid sequences that are specific to a particular pathogen. Personalised medicine requires genotyping using PCR in which the detection of one or more biomarkers, for example specific mutations, may influence clinical decisions on the nature or type of medical intervention.

PCR subjects a sample to amplification conditions in the presence of an enzyme capable of elongating nucleic acid strands, for example a polymerase. The three basic steps of a single round or thermal cycle of PCR amplification are denaturation, annealing and chain extension, each optimally taking place at different temperatures (typically 94-98° C. for denaturation; 50-65° C. for annealing, and 70-80° C. for chain extension, depending on polymerase), with each set of three steps being known by the term “thermocycling”.

### BRIEF DESCRIPTION OF THE DRAWINGS

FIG. 1 shows a perspective view of an example of a microfluidic device of the present disclosure.

FIG. 2A is a side view of an example of a microfluidic device of the present disclosure.

FIG. 2B is a side view of a further example of a microfluidic device of the present disclosure.

FIG. 3 is a side view of a further example microfluidic device of the present disclosure.

FIGS. 4A and 4B are side views of a further example microfluidic device of the present disclosure.

### DETAILED DESCRIPTION

Before particular embodiments of the present method and other aspects are disclosed and described, it is to be understood that the present method and other aspects are not limited to the particular process and materials disclosed herein as such may vary to some degree. It is also to be understood that the terminology used herein is used for the purpose of describing particular embodiments only and is not intended to be limiting, as the scope of the present method and other aspects will be defined only by the appended claims and equivalents thereof.

In the present specification, and in the appended claims, the following terminology will be used:

The singular forms “a”, “an” and “the” include plural referents unless the context clearly dictates otherwise. Thus, for example, reference to “a sensor” includes reference to one or more of such sensors.

The terms “about” and “approximately” when referring to a numerical value or range is intended to encompass the values resulting from experimental error that can occur when taking and/or making measurements.

Concentrations, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of approximately 1 wt. % to approximately 20 wt. % should be interpreted to include not only the explicitly recited concentration limits of 1 wt. % to approximately 20 wt. %, but also to include individual concentrations such as 2 wt. %, 3 wt. %, 4 wt. %, and sub-ranges such as 5 wt. % to 10 wt. %, 10 wt. % to 20 wt. %, etc.

In the following description, for purposes of explanation, numerous specific details are set forth in order to provide a thorough understanding of the present apparatus and methods. It will be apparent, however, to one skilled in the art, that the present apparatus and methods may be practiced without these specific details. Reference in the specification to “one example” or “an example” means that a particular feature, structure, or characteristic described in connection with the example is included in at least one example. The appearance of the phrase “in one example” in various places in the specification are not necessarily all referring to the same example.

Unless otherwise stated, any feature described herein can be combined with any aspect or any other feature described herein.

As used herein, the abbreviations “PCR”, “dNTPs” and “primers” refer to the “Polymerase Chain Reaction” and its components. Specifically, the term “dNTP” refers to the 2′-deoxynucleotide triphosphates used in PCR. The four standard dNTPs are 2′-deoxyadenosine 5′-triphosphate, 2′-deoxyguanosine 5′-triphosphate, 2′-deoxycytosine 5′-triphosphate and thymidine 5′-triphosphate (already lacking a 2′-hydroxyl), though modified dNTPs incorporating labels or reporter molecules, or reactive moieties may also be used. As used herein, the term “reaction mixture” refers to a mixture, for example an aqueous solution comprising all components for a PCR amplification, for example a nucleic acid sample, dNTPs, primers for a nucleic acid sequence of interest, a polymerase. Other components may include one or more salts, and a reporter molecule, such as a fluorescent reporter molecule.

As used herein, the term “primer” refers to a short single stranded nucleic acid, typically an oligodeoxynucleotide (also referred to as an oligonucleotide herein), of about 15 to 30 nucleotides in length. A primer is designed to base pair in a specific or complementary manner to a nucleic acid sequence of interest, and so is considered specific to that nucleic acid. DNA is directional, with the 3′ end of one strand forming base pairs with the 5′-end of the counter strand and a primer is usually designed so that its 5′-end base pairs to the 3′-end of the nucleic acid of interest so that DNA synthesis (which occurs in a 5′ to 3′ direction) to elongate the primer can occur.

As used herein, the terms “oligonucleotide pair”, “oligonucleotide primer pair” and “primer pair” refer to a set of two oligonucleotides that can serve as forward and reverse primers for a nucleic acid of interest. As both strands are copied and amplified in a PCR reaction, each strand requires a primer: the forward primer attaches to the start codon of the template DNA strand (the anti-sense strand), while the reverse primer attaches to the stop codon of the complementary strand of DNA (the sense strand). The 5′-end of each primer binds to the 3′-end of the complementary DNA strand of the nucleic acid of interest.

As used herein, the term “nucleic acid of interest”, or “target”, refers to a polynucleotide sequence, typically of at least one hundred, two hundred, three hundred, four hundred, five hundred or up to one thousand nucleotides in length. The polynucleotide sequence may be specific to a particular organism such as a pathogen, or may be suspected of having a particular mutation along its length, and will encode a particular polypeptide or protein, or mutant form thereof. For example, the polynucleotide sequence may encode the spike protein of SARS-CoV-2, or may encode a mutant form of the epidermal growth factor receptor (EGFR) the presence or absence of which renders a patient more or less likely to respond well to cancer treatments such as erlotinib or gefitinib.

The three basic steps of a single round or cycle of PCR amplification are denaturation, annealing and chain extension, each optimally taking place at different temperatures (typically 94-98° C. for denaturation; 50-65° C. for annealing, and 70-80° C. for chain extension, depending on polymerase), hence the term thermocycling. The denaturation step separates the two strands of double-stranded DNA, with each strand acting as a template in the later chain extension step in which a complete complementary strand to the template is produced. An oligonucleotide primer (typically comprising 15 to 30 nucleotides to ensure a balance of good specificity and efficient hybridization) is annealed to the 3′-end of each single stranded DNA molecule, and acts as a template for the synthesis of the new strand. A DNA polymerase, and a mix of dNTPs then synthesize the new strand in the chain extension step, using the original single strand of DNA as its template. Since both strands of the original DNA duplex are used as templates, a singe round or cycle of PCR results in a doubling of the number of DNA duplexes. The number of copies thus increases exponentially with the number of cycles of amplification: after 2 cycles, four DNA duplexes are present in the sample, while after 3 cycles, 8 duplexes are present.

There is a drive toward the development of portable, inexpensive microfluidic devices, which could be used for PCR at the point of care/field of use. Such devices would need rapid thermocycling, in order to amplify the nucleic acid sequences of interest and quickly provide a positive/negative test result. The vast majority of fast thermocyclers use large cooling systems or temperature baths. However, current systems for fast thermocycling are not portable nor amenable for in the field use.

The present inventors have sought to address these challenges by providing an inexpensive microfluidic device with rapid thermocycling capabilities. The proposed microfluidic device allows for rapid heating via an embedded heater and rapid cooling by providing temperature regulating flow channels in layers in thermal contact with the thermocycling chamber. Such layers may also comprise fins which provide improved heat transfer. The present inventors have also found that by providing transparent layers or lids, there is optical access for fluorescence measurement of the reaction mixture.

In one example there is provided a microfluidic device, comprising:
a microfluidic layer comprising a reaction chamber;
a temperature control module overlaying the reaction chamber; wherein the temperature control module comprises a temperature regulating flow channel disposed between two layers and in thermal contact with the reaction chamber, and wherein at least a portion of each layer comprises an optically transparent material.

In a further example there is a PCR apparatus comprising a microfluidic device as described herein.

In a further example there is provided a method of manufacturing a microfluidic device comprising;
providing a temperature control module, wherein the temperature control module comprises a temperature regulating flow channel disposed between two layers and in thermal contact with the reaction chamber, and wherein at least a portion of each layer comprises an optically transparent material; and
aligning the temperature control module onto a microfluidic layer comprising a reaction chamber so as to be in thermal contact with the reaction chamber.

In a further example there is provided a method of controlling temperature of a reaction in a microfluidic device, comprising:
introducing a reaction mixture into a reaction chamber of a microfluidic layer of a microfluidic device;
heating the reaction mixture; and
flowing a liquid through a temperature control module overlaying the microfluidic layer; wherein the temperature control module comprises a temperature regulating flow channel disposed between two layers and in thermal contact with the reaction chamber, and wherein at least a portion of each layer comprises an optically transparent material.

### Microfluidic Device

Described herein is a microfluidic device. The device comprises a microfluidic device, comprising a microfluidic layer comprising a reaction chamber; a temperature control module overlaying the reaction chamber; wherein the temperature control module comprises a temperature regulating flow channel disposed between two layers and in thermal contact with the reaction chamber, and wherein at least a portion of each layer comprises an optically transparent material.

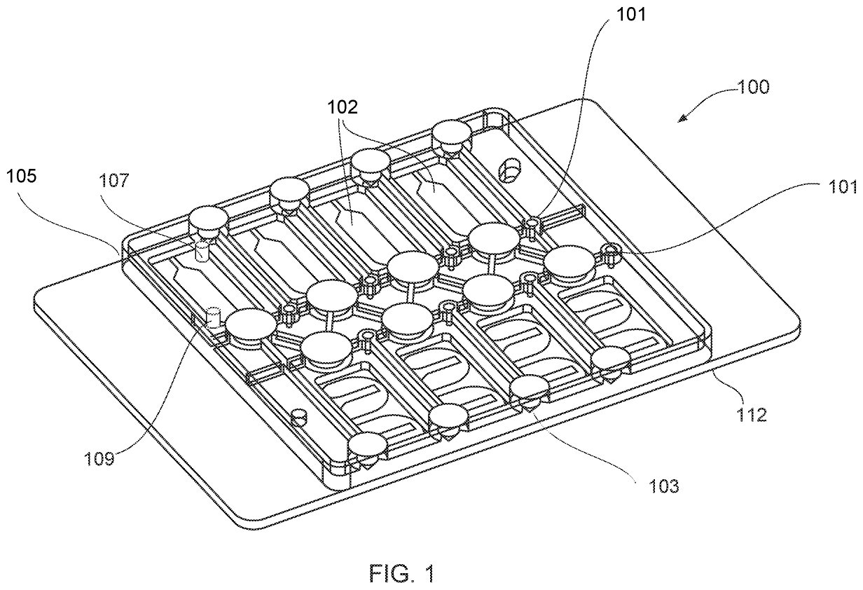

FIG. 1 shows a perspective view of a microfluidic device 100 in accordance with the disclosure. Generally, microfluidic device 100 comprises a substrate 112 on which a reaction chamber 102 is provided. In the example of FIG. 1, eight reaction chambers are provided: four elongate chambers in the top row, and four serpentine reaction chambers in the bottom row. Reaction chamber 102 forms part of a microfluidic layer 105 of microfluidic device 100. Each reaction chamber 102 is provided with a dedicated fluid inlet or port 101, and a dedicated vent or exhaust 103. As can be seen in the first elongate reaction chamber on the left, a fluid inlet 107 and a fluid outlet 109 are provided, for respectively providing and removing a temperature regulating liquid into a temperature regulating flow channel of a temperature control module forming part of microfluidic layer 105. The structure of the microfluidic layer will now be described in more detail with reference to the remaining Figures.

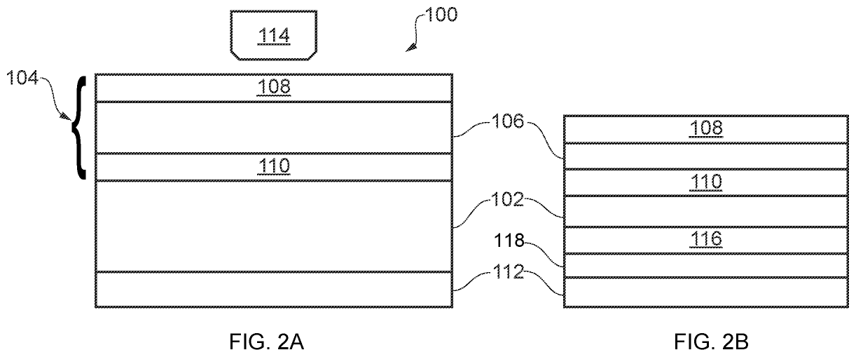

FIG. 2A shows a side view of a microfluidic device 100 in accordance with the disclosure. The microfluidic device 100 comprises a reaction chamber 102, which may hold the reaction mixture. The reaction chamber 102 forms part of a microfluidic layer of the microfluidic device. The microfluidic device further comprises a temperature control module 104, which comprises a temperature regulating flow channel 106 disposed between two layers, an outer layer 108 and an inner layer 110. The temperature control module 104 overlays the microfluidic layer such that inner layer 110 is in thermal contact with a reaction mixture in the reaction chamber 102. In some examples, temperature control module 104 forms part of microfluidic layer 105. The microfluidic device further comprises a substrate 112. FIG. 2A also shows an optical sensor 114 configured to obtain optical signals from the reaction mixture through both the outer layer 108 and the inner layer 110 with temperature regulating flow channel 106 disposed therebetween.

FIG. 2B shows another example of a microfluidic device 100 as described above, however in this example, the microfluidic device 100 comprises a second inner layer 116. The arrangement of the device in FIG. 2B is such that the outer layer 108 and the first inner layer 110 define a first temperature regulating channel 106. The second inner layer 116 and substrate 112 define a second temperature regulating channel 118. In this example, the microfluidic device 100 comprises a first temperature regulating channel 106 positioned above the reaction chamber 102 and a second temperature regulating channel 118 positioned below the reaction chamber 102.

In some examples, substrate 112 may be formed from any material suitable for microfluidics, such as glass, silicon, SU-8 (an epoxy-based photoresist material), or polycarbonate. In some examples, a heater is provided on or within the substrate, to provide heat to the reaction chamber. In some examples, the substrate comprises or is a printed circuit board (PCB), and so in some examples is termed a PCB substrate. In some examples, the heater comprises one or more printed electrical traces on a substrate to provide heat to the reaction chamber. In some examples, the heater is provided above or below the plane of the microfluidic device. In some examples, the heater is embedded into a substrate on which the reaction chamber is disposed. The heater may be embedded into the substrate by machining or etching portions of the substrate into which the heater can be embedded, or by encapsulating the heater in a liquid precursor material that can be solidified or cured to form the substrate. A suitable material is SU-8, which is a liquid, until cured by UV light. In other examples, the heater is provided on a surface of the substrate. In some examples, the heater comprises a flat panel heater or one or more thermally conductive printed electrical traces. In some examples, the heater comprises a Peltier device, a flat panel heater in the form of a solid-state active heat pump. In some examples, the heater receives electrical power from electrically conductive wires provided on or to the microfluidic device to form an electrical circuit which supplies electrical current to the heater. Such components may be controlled by a controller located on or off the microfluidic device via control signals.

In some examples, a dielectric layer is disposed over the substrate and/or a heater embedded in or on the substrate. The dielectric layer may be spun on or sputtered onto the substrate. Thus, in some examples, the substrate comprises a dielectric coating of polyimide, SU-8, silicon oxide, silicon nitride, aluminium oxide, aluminium nitride or any combination/stack thereof. Another suitable material is Kapton®, which may be incorporated into a coating or stack with any of the aforementioned materials.

In some examples, reaction chamber 102 is provided in a microfluidic layer 105 (also termed a microfluidic stack) of the microfluidic device, disposed on substrate 112. As used herein, the terms “microfluidic layer”, “microfluidic stack”, “fluidic layer” or “fluidic stack” refer to the components of the microfluidic device through which one or more fluids can pass during use of the microfluidic device, for example through one or more microfluidic channels and chambers. The terms are intended to encompass multiple flow paths, for example in different levels of the layer/stack, and distinguish these flow channel-containing components from other operational modules such as electronic circuitry and sensors. In some examples, microfluidic layer 105 includes temperature control module 104. In other examples, the temperature control module 104 is arranged on microfluidic layer 105, so as to be in thermal contact with the reaction chamber 102. The microfluidic layer or microfluidic stack may comprise any material or combination of materials suitable for use in microfluidic devices, including polycarbonate, and cyclic olefin copolymer (COC). In some examples, reaction chamber 102 is formed in a microfluidic layer or microfluidic stack by moulding, or selectively etching or machining away regions of material so as to form a reaction chamber. In some examples, reaction chamber 102 is formed wholly within the material forming the microfluidic layer (for example COC), with that material also forming the base of the reaction chamber. In other examples, the material forming the microfluidic layer forms the walls of the reaction chamber, with the underlying substrate (for example with a dielectric layer as described) forming the base or floor of the reaction chamber. Other layers present in a microfluidic stack may include layers of adhesive to bond the microfluidic layer to the substrate and/or bond layers of a microfluidic stack to each other. Suitable adhesives include pressure-sensitive adhesives, which typically comprise an elastomer based on acrylic, silicone or rubber optionally compounded with a tackifier such as a rosin ester. Convenient pressure sensitive adhesives are in the form of double-sided films or tape, such as the acrylic adhesives 200MP and 7956MP available from 3M™. In some examples, the microfluidic layer is provided with one or more fluid inlets and outlets to provide a liquid such as a reaction liquid to the or each reaction chamber. In some examples, the or each reaction chamber is also provided with a vent. The presence of a vent enables unhindered flow of liquid through the reaction chamber and minimises risk of unwanted bubble formation within the reaction chamber.

The microfluidic device comprises a temperature control module overlaying the reaction chamber and comprising a temperature regulating flow channel disposed between two layers and in thermal contact with the reaction chamber. In some examples, the microfluidic layer or microfluidic stack comprises or includes the temperature control module. In some examples, the temperature control module comprises a second temperature regulating flow channel, wherein the second temperature regulating channel is disposed below the reaction chamber. The temperature regulating flow channels allow for the flow of a fluid therein to assist with heating or cooling of the reaction mixture in the reaction chamber.

In some examples, the temperature regulating flow channel is formed in a material such as SU-8, cyclic olefin copolymer (COC), or polycarbonate (PC) sandwiched between the layers which define the upper and lower boundaries of the temperature regulating flow channel. As described above in connection with the microfluidic layer and reaction chamber, the temperature regulating flow channel may be formed by moulding, etching or micromachining. Any configuration or geometry for the temperature regulating flow channel that maximises heat transfer from the reaction chamber to the temperature regulating flow channel is possible. In one example, the temperature regulating flow channel has a cross-sectional geometry that is substantially identical to that of the reaction chamber. In other examples, the temperature regulating flow channel has a larger width and/or length than the reaction chamber, to increase the volume of fluid that can be present in the temperature regulating flow channel at any one time. Increasing the volume of fluid in the temperature regulating flow channel increases the efficiency of heat transfer from the reaction chamber.

In some examples, the temperature regulating flow channel is provided with an inlet port, or fluid inlet, and an outlet port, or fluid outlet. In some examples, inlet and outlet ports are configured to provide a flow of liquid, for example a cooling liquid, to and from the temperature regulating flow channel.

In some examples, the layers defining the temperature regulating flow channel comprise or consist of a thermally conductive material. In some examples, at least the layer of the temperature regulating flow channel that is immediately disposed above the reaction chamber comprises or consists of a thermally conductive material.

In some examples, a portion of each of the layers defining a temperature regulating flow channel comprises a transparent material that is also thermally conductive. In some examples, each of the layers defining the temperature regulating flow channel consists of an optically transparent material that is also thermally conductive. In some examples, the layers (also described herein as lids) comprise or consists of an optically transparent polymer or an optically transparent high thermal conductivity ceramic. Some examples of optically transparent polymers may include cyclic olefin copolymer (COC), and polycarbonate (PC) sheets (such as Lexan). In some examples, the layers comprise or consist of thin layers of glass, such as glass plates machined with metal plated through holes for improved heat transfer, or layers of diamond. Some examples of optically transparent high thermal conductivity ceramic may include aluminium oxide, aluminium nitride, zirconia and silicon carbide. Other materials which are optically transparent may also be used. The use of a transparent material allows for optical access to the reaction mixture in the reaction chamber for testing. In some examples, the testing may involve the optical detection of the fluorescence of one or more reporter molecules that permit monitoring of a reaction by optical means. In some examples, the refractive index of the optically transparent material may be equal to, or similar to, the refractive index of the fluid in the temperature regulating flow channel in order to avoid scattering of the light or optical losses from the interfaces. The various layers of the temperature control module may be bonded together using adhesive layers, such as a pressure sensitive film as described above, though other bonding methods such as liquid adhesives are also suitable.

In some examples, each layer or lid may have a thickness of from 1 μm to 1 mm, for example from 1 μm to 500 μm, for example from 1 μm to 250 μm, for example from 10 μm to 100 μm, for example about 100 μm. In some examples, the thickness of the outer layer differs from the thickness of the inner layer. By having an inner layer of the temperature control module which is in contact with the reaction chamber and which is thinner than the outer layer, a greater thermal transfer from the reaction chamber to the fluid in the temperature regulating flow channel can be achieved.

In some examples, each layer forming the temperature control module may comprise an optically opaque material which further comprises a window which comprises an optically transparent material. The optically transparent material forming the window may comprise a material already described herein. The optically opaque material may comprise an opaque polymer, a metal, or a silica filled epoxy such as those described above. By only having a portion of the layers, e.g. a window, as the optically transparent material, high thermal conductivity materials such as copper or aluminium can be used as optically opaque materials in the layers in which the window is provided, thus increasing the efficiency of heat transfer from the reaction chamber.

In some examples, the substrate of the microfluidic device comprises a PCB substrate. In some examples, the PCB substrate is provided with embedded or printed electrical traces for heating and temperature sensing. In some examples, an electrical trace of the PCB substrate is a heater and performs the function of heating the reaction mixture in the reaction chamber.

In some examples, the microfluidic device further comprises an optical sensor configured to obtain optical signals from a reaction mixture in the reaction chamber through the optically transparent material. In some examples, such as shown in the microfluidic device of FIG. 2A, optical sensor 114 is a separate unit to microfluidic device 100 and is spaced apart from upper layer 108 of the temperature control module 104. In some examples, the optical sensor is an integral component of the microfluidic device and is embedded into the microfluidic device. For example, the optical sensor may be embedded into upper layer 108 of the temperature control module.

In some examples, at least one of the layers of the temperature control module comprises at least one fin which extends from at least one layer into the temperature regulating flow channel to provide enhanced heat transfer to or from the reaction mixture. In some examples, at least one of the layers of the temperature control module comprises at least one fin which extends from the at least one layer into the temperature regulating flow channel and into the reaction chamber to provide enhanced heat transfer to or from the reaction mixture. In some example, there may be a plurality of fins which extend from one or more of the layers of the microfluidic device.

In some examples, the at least one fin or the plurality of fins may extend fully from the outer layer to the substrate. In some examples, the at least one fin or the plurality of fins may extend from the substrate into the reaction chamber to increase thermal transfer from the substrate to a reaction mixture within the reaction chamber. In some examples, the at least one fin or the plurality of fins may extend only partially into the temperature regulating channel. In some examples, the at least one fin or the plurality of fins may extend partially or fully into the reaction chamber. In some examples, the at least one fin or the plurality of fins extend sufficiently far into the reaction chamber so as to contact a reaction mixture in the chamber. In some examples, the plurality of fins forms a pattern, for example, a square grid. In some examples, the at least one fin or the plurality of fins may project from a plurality of surfaces to maximise heat transfer to or from a reaction mixture in the reaction chamber. In some examples, the at least one fin has or the plurality of fins have a cross-sectional geometry that is circular, square, rhomboid, elliptical or any other geometry. In some examples, the at least one fin has or the plurality of fins have a cross-sectional geometry that resembles an airfoil, oriented along a flow axis of the temperature regulating channel. Use of such airfoil fins reduces drag of the temperature controlling liquid flowing through the temperature regulating channel, allowing for faster flow rates and quicker cooling.

In some examples, the plurality of fins are spaced apart to provide for optical interrogation of a reaction mixture in the reaction chamber. In some examples, the fins are spaced apart at regular intervals to allow for optical interrogation of a reaction mixture in the reaction chamber.

In some examples, the at least one fin is comprised of a high thermal conductivity material. Some examples of high thermal conductivity materials include transition metals, for example copper or aluminium. In some examples, the at least one fin is comprised of a stainless steel, or other metal alloy having high thermal conductivity. In some examples, a plurality of fins may be provided and they all comprise or consist of the same high thermal conductivity material. In some examples, the plurality of fins may comprise or consist of different high thermal conductivity materials.

In some examples, the at least one fin comprises at least one polished surface to increase the reflectivity of the fin and improve the optical sensor readout. In some examples, the at least one fin or at least one surface of the reaction chamber comprises a reflective coating to increase reflectivity of the reaction chamber and improve the optical sensor readout. Examples of reflective coatings include aluminium, silver or gold coatings, which may themselves have a protective dielectric overcoat of a transparent material such as silica or alumina to protect them from oxidation. Other examples of reflective coatings include diffuse reflective materials such as barium sulfate. In some examples, a reflective coating is provided onto a surface via a sputtering, spraying or evaporation process.

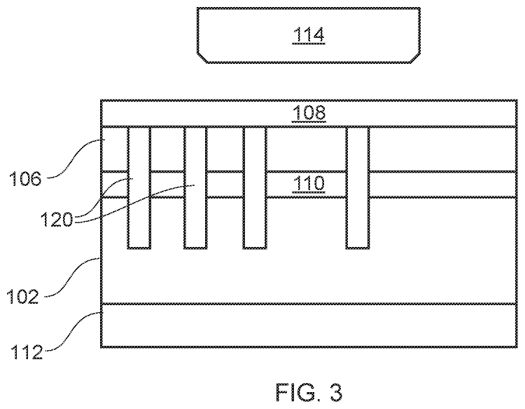

FIG. 3 shows a side view of a microfluidic device which comprises a plurality of fins 120 which extend from the outer layer 108. In FIG. 3, the fins 120 extend from the outer layer 108, through the temperature regulating flow channel to the reaction volume (also known as the reaction mixture) to improve heat transfer. The outer layer 108 and the inner layer 106 of the temperature control module are formed from an optically transparent material so that an optical reading may be taken by the optical sensor 114.

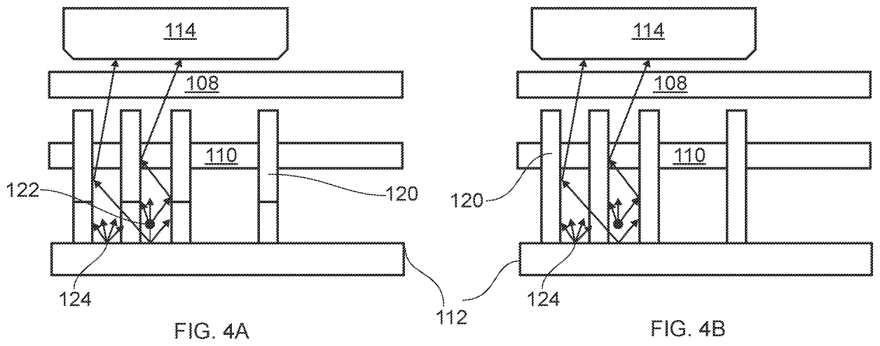

FIG. 4A shows a side view of a microfluidic device where a plurality of fins 120 extend partially across the reaction mixture (reaction volume) and the temperature regulating flow channel, in this case extending upwards from substrate 112. FIG. 4A shows the path of fluorescence 122, produced in a reaction mixture, travelling through the optically transparent inner layer 110 and outer layer 108, to the optical sensor. FIG. 4A also shows a reflective coating 124 applied to the top surface of the substrate 112 to improve reflectivity.

FIG. 4B is similar to FIG. 4A except that it also shows a plurality of fins extending fully from substrate 112 across the reaction mixture to the temperature regulating flow channel.

In some examples, an optical sensor is provided, and configured to obtain optical signals from the reaction chamber. In some examples, the optical sensor is a fluorescence detector and the optical signals are fluorescence signals. In some examples, the optical sensor comprises a light source and a detector, wherein the light source is for example a laser diode, or an LED, configured to emit light of a wavelength suitable to cause fluorescence of a fluorescent reporter molecule in the reaction mixture. In some examples, the detector may be a charge coupled device (CCD) or pin photodiode to detect the emitted fluorescent light. In some examples, the optical sensor is arranged above or below the thermocycling chamber, for example above or below a plane in which the liquid sample is being thermocycled. In some examples, the microfluidic device is provided with an optical window or opening that allows transmission of light therethrough to an optical sensor located in an apparatus external to the microfluidic device, or within the microfluidic device itself. In some examples, the optical sensor is embedded into a lid or cover of the microfluidic device.

In some examples, a method of manufacturing a microfluidic device is described, wherein the method comprises:
providing a temperature control module, wherein the temperature control module comprises a temperature regulating flow channel disposed between two layers and in thermal contact with the reaction chamber, and wherein at least a portion of each layer comprises an optically transparent material; and
aligning the temperature control module onto a microfluidic layer comprising a reaction chamber so as to be in thermal contact with the reaction chamber.

In some examples, the reaction chamber may be formed in the microfluidic layer by etching or micromachining the reaction chamber into a material forming at least part of the microfluidic layer or into a surface of a material forming at least part of the microfluidic layer. In some examples, forming the reaction chamber may comprise forming the reaction chamber in a microfluidic layer, and arranging the microfluidic layer comprising the reaction chamber on a substrate, with the substrate forming the floor of the reaction chamber. In some examples, the microfluidic layer may be bonded to the substrate by any suitable means, for example using an adhesive such as a pressure sensitive adhesive as described previously.

In some examples, the temperature control module comprises a discrete module that is manufactured separately to the microfluidic layer. The temperature control module may be provided by forming a temperature regulating flow channel in a layer of material and arranging this layer between the two layers of the temperature control module. The temperature control module may then be aligned onto the microfluidic layer so as to be in thermal contact with the reaction chamber.

In some examples, the temperature control module forms part of the microfluidic layer, in a microfluidic stack, and is formed by sequential addition of layers, for example adding the material comprising the microfluidic layer onto the substrate (with any bonding layer therebetween), bonding the first layer of the temperature control module to the microfluidic layer, bonding the material forming the temperature regulating flow channel to the first layer, and bonding the second layer to the material forming the temperature regulating flow channel. It will be understood that any necessary machining of layers, for example the microfluidic layer or the layer of material forming the temperature regulating flow channel can be performed before or after the layer in question has been added to the underlying layer.

In some examples, the method of manufacturing further comprises providing at least one fin which extends from at least one layer into the temperature regulating flow channel. In some examples, the method of manufacturing further comprises providing at least one fin which extends from at least one layer into the temperature regulating flow channel and into the reaction chamber. In some examples, providing at least one fin comprises providing at least one through-hole in a layer defining the temperature regulating flow channel using, for example, a laser, and arranging the at least one fin in the at least one through-hole. In some examples, the at least one through-hole is formed in the layer by casting a liquid precursor material into a mould and forming the layer of material having at least one through-hole by solidifying or curing the liquid precursor material. In some examples, an adhesive such as an epoxy adhesive is used to seal the at least one fin in the at least one through-hole.

In some examples, the method comprises providing an optical sensor configured to obtain optical signals from the reaction chamber. In some examples, the optical sensor is a fluorescent sensor. In some examples, the optical sensor is directly integrated into the microfluidic device, for example into a wall or cover of the microfluidic or is located elsewhere in the apparatus but configured to receive signals from the reaction chamber.

In some examples, the microfluidic device described herein forms part of a PCR apparatus and the reaction chamber is a thermocycling chamber. In some examples, the microfluidic device comprises a plurality of reaction chambers and therefore a plurality of thermocycling chambers. In some examples, the microfluidic device is in the form of a cassette, or chip, to be used in the PCR apparatus. In some examples, the microfluidic device may be a single use or disposable device. In some examples, the microfluidic device may be configured to be inserted into or received by a port in the apparatus.

In some examples, the microfluidic device may be provided with one or more fluidic connections that are configured to engage with one or more corresponding fluidic connections in the apparatus, to enable fluid flow from the apparatus into the microfluidic device, for example to enable transfer of a sample injected into an injection port of the apparatus to be transferred to the reaction chamber of the microfluidic device. In other examples, the or each reaction chamber of the microfluidic device may be filled with sample prior to inserting the microfluidic device into the apparatus, for example by manual pipetting a sample solution through an inlet port such as a Luer connector or membrane valve.

In some examples, the microfluidic device may be provided with one or more fluidic connections that are configured to engage with one or more corresponding fluidic connections in the apparatus, to enable fluid flow from the apparatus into the temperature regulating flow channel of the microfluidic device.

In some examples, the PCR apparatus comprises an electrical interface, configured to contact an electrical interface provided on the microfluidic device. The electrical interface on the microfluidic device may be coupled to any component of the device that requires electrical current to operate. Examples of such devices include heater elements, either in flat panel form or printed conductive trace form, and actuators for controlling fluid flow within the microfluidic device. In some examples, the electrical interfaces may be multi-pin input/output off board connectors, for example 44-pin connectors that enable electrical coupling of the microfluidic device to a computer module of the PCR apparatus. Each pin of the electrical interface may provide an electrical contact to a specific component of the microfluidic device, such as the individually addressable or controllable heaters described herein. The electrical coupling of the device to the apparatus allows control signals from the computer module to be sent to the device so that electrical current can be sent to desired modules of the device.

As noted above, the PCR apparatus may comprise a computer control module. In some examples, the computer control module comprises a processor comprising hardware architecture to retrieve executable code from a data storage device or computer-readable medium and execute instructions in the form of the executable code. The processor may include a number of processor cores, an application specific integrated circuit (ASIC), field programmable gate array (FPGA) or other hardware structure to perform the functions disclosed herein. The executable code may, when executed by the processor, cause the processor to implement the functionality of one or more hardware components of the device and/or apparatus such as one or more heaters and/or one or more optical detectors. In the course of executing code, the processor may receive input from and provide output to a number of the hardware components, directly or indirectly. The computer control module may communicate with such components via a communication interface which may comprise electrical contact pads, electrical sockets, electrical pins or other interface structures. In one example, the communication interface may facilitate wireless communication.

In some examples, the computer control module facilitates the introduction of a sample into the reaction chamber, or into multiple reaction chambers. For example, the computer control module may control a series of valves and pumps in the apparatus or on the microfluidic device to direct flow of a test sample or solution to the reaction chamber.

In some examples, the computer control module may further control the processing of a sample in a reaction chamber, for example by subjecting the reaction chamber to thermocycling conditions. For example, the computer control module may control, through the output of control signals, the operation of one or more heaters to control the temperature and duration of heating within the or each reaction chamber, or the operation of an optical sensor. As a result, a sample may undergo various selected reactions, various selected heating cycles and various sensing operations under the control of the computer control module.

### Method of Controlling Temperature of a Reaction in a Microfluidic Device

In some examples, there is provided a method of controlling temperature of a reaction in a microfluidic device, comprising:
introducing a reaction mixture into a reaction chamber of a microfluidic layer of a microfluidic device;
heating the reaction mixture; and
flowing a liquid through a temperature control module overlaying the microfluidic layer; wherein the temperature control module comprises a temperature regulating flow channel disposed between two layers and in thermal contact with the reaction chamber, and wherein at least a portion of each layer comprises an optically transparent material.

In some examples, the method of controlling temperature further comprises:
(a) providing a temperature regulating fluid in the temperature regulating flow channel with a lower temperature than the reaction mixture to cool the reaction mixture, or
(b) providing a temperature regulating fluid in the temperature regulating flow channel with a higher temperature than the reaction mixture to heat the reaction mixture.

In some examples, the fluid in the temperature regulating channel is deionized water, propylene glycol or polyalkylene glycol, or any other colorless liquid that has a high heat capacity and low viscosity, and is chemically inert. In some examples, the cooling or heating is achieved by flowing fluid along the temperature regulating flow channel. In some examples, the fluid is static in the temperature regulating flow channel. By providing a fluid in the temperature regulating flow channels that is either cooler or hotter than the reaction mixture in the reaction chamber, the temperature of the reaction mixture may be regulated.

In some examples, the method further comprises providing at least one fin which extends from at least one of the layers into the temperature regulating flow channel and reaction mixture to provide enhanced heat transfer.

In some examples, the microfluidic device may form part of a PCR apparatus. In some examples, the reaction mixture is a liquid volume containing PCR reactants and reagents. In some examples the reaction mixture is introduced into the reaction chamber which may also be a thermocycling chamber. In some examples, the reaction mixture introduced into the reaction chamber/thermocycling chamber comprises an aqueous solution of PCR reactants and reagents. In some examples, the liquid volume containing a PCR mixture comprises a nucleic acid sample of interest, to be amplified, one or more pairs of PCR primers complementary to a nucleic acid sample of interest, a polymerase, dNTPs and salts such as MgCl₂. Suitable polymerases include the thermostable polymerases Taq, Bst and Pfu. In some examples, the test solution comprises the four standard dNTPs, i.e. dGTP, dCTP, dATP and TTP. In some examples, the liquid volume also contains one or more reporter molecules that permit monitoring of the amplification by optical means. In some examples, the one or more reporter molecules comprise non-specific fluorescent dyes, such as SYBR Green, which intercalates into any double-stranded DNA, leading to an increase in fluorescence as more double-stranded DNA is produced. Other reporter molecules include target-specific fluorescent reporter molecules, such as the TaqMan hydrolysis probes of target-specific nucleic acids labelled with fluorescent reporter and quencher, with the probe being hydrolyzed by the exonuclease activity of the Taq polymerase, releasing the reporter from the quencher and again leading to an increase in fluorescence, and the Scorpion® probes, in which the fluorescent reporter is covalently linked to a primer and additional sequence (complementary to a sequence of the nucleic acid of interest, forming a hairpin-loop conformation bringing the fluorescent dye into proximity to a quencher. After one round of chain extension, the sequence of the hairpin-loop opens up, base-pairs with the newly formed sequence and spaces the fluorescent reporter and quencher from one another, enabling the reporter to fluoresce when excited.

In some examples, the reaction mixture may be prepared by combining the nucleic acid sample, first and second oligonucleotides forming a primer pair complementary to the nucleic acid of interest, the dNTPs, polymerase and buffer/salts.

In some examples, the reaction mixture comprises a plurality of oligonucleotide or primer pairs, each complementary to a different nucleic acid of interest. In these examples, a multiplexed PCR analysis is enabled.

In some examples, the liquid volume of the reaction mixture has a volume of less than 200 μL, for example less than 150 μL, for example less than 100 μL, for example less than 50 μL, for example about 5 μL. In some examples, the liquid volume has a volume of greater than 5 μL, for example greater than 50 μL, for example greater than 100 μL, for example greater than 150 μL, for example about 200 μL. In some examples, the liquid volume of the reaction mixture is sufficient to completely fill the reaction chamber and be in direct contact with the temperature control module overlaying the microfluidic layer. In some examples, the liquid volume of the reaction mixture is sufficient to completely fill the reaction chamber and be in direct contact with the lower layer of the temperature control module overlaying the microfluidic layer. In some examples, the liquid volume of the reaction mixture is sufficient to be in direct contact with at least one fin which is comprised within the temperature control module overlaying the microfluidic layer and which extends into the reaction chamber.

In some examples, the test solution comprises a nucleic acid sample obtained from a subject. In some examples, the nucleic acid sample may comprise a nucleic acid for analysis and is to be amplified in a method as described herein. In some examples, the nucleic acid sample may comprise a plurality of nucleic acids for analysis which are to be amplified in a method as described herein. In some examples, the test solution is suspected of comprising one or a plurality of nucleic acid sequences of interest. In some examples, the nucleic acid sample is obtained from one or more of a blood sample, a tissue sample, a saliva sample or mucosal sample. In some examples, the nucleic acid sample is obtained using a swab. In some examples, the nucleic acid sample is isolated from the bodily fluid or tissue via which it was obtained. In some examples, the nucleic acid sample is not isolated from the bodily fluid or tissue via which it was obtained. In some examples, the nucleic acid sample obtained from a subject is incorporated into a test solution with or without any isolation or preparation. In some examples, the nucleic acid sample obtained from a subject is dissolved or dispersed in an aqueous solution, thus forming a test solution. In some examples, a primer pair complementary to the nucleic acid sequence of interest, a polymerase, and mix of dNTPs may also be added to the test solution before or after the nucleic acid sample has been dissolved or dispersed. The polymerase and dNTPs may be added to the test solution as part of a PCR “Master Mix”, before or after the nucleic acid sample has been dissolved or dispersed. A PCR Master Mix is a mixture of PCR reagents, already at optimized concentrations, which can be readily aliquoted and added to the test solution. The Master Mix usually comprises the DNA elongation enzyme (e.g. a polymerase), the dNTPs, MgCl₂ as an enzyme co-factor (although other co-factors, such as MgSO₄ may be used with certain enzymes), all dissolved in an aqueous buffer. The Master Mix may also include a reporter molecule, such as a fluorescent dye as described herein. The LightCycler® 480 SYBR Green I Master Mix includes a polymerase, co-factor, dNTPs and SYBR Green I in a buffered solution, meaning that only the nucleic acid sample (and, if appropriate, a primer) need to be added. However, the reporter molecule may also be added separately.

Once the liquid volume has been introduced into the reaction chamber, heat is provided to raise the temperature of the reaction mixture. In some examples, heat is provided by means of a heater, for example a printed electrical trace provided in or on the substrate on which the reaction chamber is located. The temperature regulating flow channel will also be filled with fluid, for example water. When the temperature of the reaction mixture needs to be adjusted, the temperature regulating flow channels will be provided with a fluid which is either hotter or cooler than the reaction mixture to either heat or cool the reaction mixture as appropriate, in addition to the control of the heater. In some examples, the fluid is flowed along the temperature regulating flow channel. In some examples, the fluid is flowed along the temperature regulating channel at a flow rate of less than 100 μL/sec, for example less than 75 μL/sec, for example less than 50 μL/sec, for example less than 25 μL/sec, for example less than 20 μL/sec, for example less than 10 μL/sec, for example about 5 μL/sec. In some examples, the fluid is flowed along the temperature regulating channel at a flow rate of greater than 5 μL/sec, for example greater than 20 μL/sec, for example greater than 30 μL/sec, for example greater than 50 μL/sec, for example greater than 70 μL/sec, for example greater than 85 μL/sec, for example greater than 90 μL/sec, for example about 100 μL/sec. While greater, or quicker, heat transfer may be achieved using a flow of fluid along the temperature regulating channel, adequate heat transfer may also be achieved without any fluid flow. Thus, in some examples, the fluid is static in the temperature regulating flow channel. In some examples, the temperature regulating flow channels work in combination with a heater provided on a substrate to heat the reaction mixture.

In some examples, the method comprises performing one or more rounds of PCR amplification with the reaction mixture prior to any optical detection such as fluorescence detection.

For example, the three basic steps of a single round of PCR amplification are denaturation, annealing and chain extension, each optimally taking place at different temperatures (typically 94-98° C. for denaturation; 50-65° ° C. for annealing, and 70-80° C. for chain extension, depending on polymerase). A reaction mixture present in the reaction chamber of the microfluidic device described herein may therefore be heated, for example by a heater in the microfluidic device to a denaturation temperature of from 94-98° C. for a sufficient time for any double stranded DNA to separate or denature into single stranded DNA. According to the thermocycling protocol described above, the reaction mixture must then be cooled to an annealing temperature of from 50-65° C. While the turning off of a heater in thermal contact with the reaction mixture will cease any further heating, it will not rapidly cool the reaction mixture, as is desirable. However, with the introduction of a temperature control module as described herein, heat can be dissipated away from the reaction mixture to a fluid (for example water) flowing through the temperature control module. The use of one or more fins can further enhance heat transfer as described.

While the apparatus, methods and related aspects have been described with reference to certain examples, it will be appreciated that various modifications, changes, omissions, and substitutions can be made without departing from the spirit of the disclosure. It is intended, therefore, that compositions, methods and related aspects be limited only by the scope of the following claims. Unless otherwise stated, the features of any dependent claim can be combined with the features of any of the other dependent claims, and any other independent claim.

1. A microfluidic device, comprising:
a microfluidic layer comprising a reaction chamber; and
a temperature control module overlaying the reaction chamber; wherein the temperature control module comprises a temperature regulating flow channel disposed between two layers and in thermal contact with the reaction chamber, and wherein at least a portion of each layer comprises an optically transparent material.

2. The microfluidic device according to claim 1, where each layer consists of an optically transparent material.

3. The microfluidic device according to claim 1, wherein each layer comprises an optically opaque material which further comprises a window which comprises an optically transparent material.

4. The microfluidic device according to claim 1, wherein the temperature control module further comprises a second temperature regulating flow channel, wherein the second temperature regulating channel is disposed below the reaction chamber.

5. The microfluidic device according to claim 1, wherein the optically transparent material comprises an optically transparent polymer or an optically transparent high thermal conductivity ceramic.

6. The microfluidic device according to claim 1, wherein at least one of the two layers comprises at least one fin which extends from at least one layer into the temperature regulating flow channel and reaction chamber to provide enhanced heat transfer.

7. The microfluidic device according to claim 6, wherein at least one of the two layers comprises a plurality of fins, and wherein the plurality of fins are spaced apart to allow for optical interrogation of a reaction mixture in the reaction chamber.

8. The microfluidic device according to claim 6, wherein the at least one fin is comprised of a high thermal conductivity material.

9. The microfluidic device according to claim 1, wherein the microfluidic device further comprises a PCB substrate with embedded traces for heating and temperature sensing.

10. The microfluidic device according to claim 1, wherein the microfluidic device further comprising an optical sensor configured to obtain optical signals through the optically transparent material.

11. A method of manufacturing a microfluidic device, comprising:
providing a temperature control module, wherein the temperature control module comprises a temperature regulating flow channel disposed between two layers and in thermal contact with the reaction chamber, and wherein at least a portion of each layer comprises an optically transparent material; and
aligning the temperature control module onto a microfluidic layer comprising a reaction chamber so as to be in thermal contact with the reaction chamber.

12. The method of manufacturing according to claim 11, wherein the method further comprises providing at least one fin which extends from at least one layer into the temperature regulating flow channel and reaction chamber to provide enhanced heat transfer.

13. A method of controlling temperature of a reaction in a microfluidic device, comprising:
introducing a reaction mixture into a reaction chamber of a microfluidic layer of a microfluidic device;
heating the reaction mixture; and
flowing a liquid through a temperature control module overlaying the microfluidic layer; wherein the temperature control module comprises a temperature regulating flow channel disposed between two layers and in thermal contact with the reaction chamber, and wherein at least a portion of each layer comprises an optically transparent material.

14. The method of controlling temperature according to claim 13, wherein the method further comprises:
(a) providing a temperature regulating fluid in the temperature regulating flow channel with a lower temperature than the reaction mixture to cool the reaction mixture, or
(b) providing a temperature regulating fluid in the temperature regulating flow channel with a higher temperature than the reaction mixture to heat the reaction mixture.

15. The method of controlling temperature according to claim 13, wherein the method further comprises providing at least one fin which extends from at least one of the layers into the temperature regulating flow channel to provide enhanced heat transfer.
